(19) United States
(12) Patent Application Publication
(10) Pub. No.: US 2026/0041575 A1
(43) Pub. Date: Feb. 12, 2026

(54) RAPID EXCHANGE STENT DELIVERY DEVICE

(71) Applicant: Micro-Tech (Nanjing) Co., Ltd., Nanjing (CN)

(72) Inventors: Yaoyuan Wang, Nanjing (CN); Jianyu Wei, Nanjing (CN); Hongyan Jin, Nanjing (CN); Jie Hu, Nanjing (CN); Yu Ji, Nanjing (CN); Changqing Li, Nanjing (CN)

(21) Appl. No.: 19/293,021

(22) Filed: Aug. 7, 2025

(30) Foreign Application Priority Data
Aug. 8, 2024 (CN) 202411087412.3
Jul. 28, 2025 (CN) 202511047172.9

(51) Int. Cl. A61F 2/966; A61F 2/95

(52) U.S. Cl. CPC A61F 2/966; A61F 2/9517; A61F 2002/9511; A61F 2002/9528

(57) ABSTRACT

A rapid exchange stent delivery device is provided that relates to the technical field of stent delivery. The rapid exchange stent delivery device releases a stent toward a distal end along a guide wire, and the delivery device includes: an outer tube and an inner tube inserted inside the outer tube; a guide wire inlet is arranged on a distal end of the inner tube, and a guide wire outlet is arranged on a side wall; and the outer tube has an avoiding part extending toward the distal end to avoid and guide out the guide wire extending from the guide wire outlet, so that the outer tube can move relative to the inner tube, and the inner tube pushes the stent out of the distal end of the outer tube for release.

### CROSS-REFERENCE TO RELATED APPLICATION

The present disclosure claims priority to Chinese Patent Application No. 202411087412.3, filed with the Chinese Patent Office on Aug. 8, 2024, entitled “RAPID EXCHANGE STENT DELIVERY DEVICE”; and Chinese Patent Application No. 202511047172.9, filed with the Chinese Patent Office on Jul. 28, 2025, entitled “RAPID EXCHANGE STENT DELIVERY DEVICE”, the entire contents of which are incorporated herein by reference.

### TECHNICAL FIELD

The present disclosure relates to the technical field of stent delivery, and in particular to a rapid exchange stent delivery device.

### BACKGROUND ART

Usually, the guide wire of the coated stent or bare stent needs to extend to and protrude from the handle end. The guide wire extends along the outer tube for a long distance, and the operation duration of the surgery is relatively long, which makes it difficult to improve the efficiency of surgery. If the guide wire is led out from the guide wire outlet located near the distal end on the side wall of the inner tube, an opening on the side wall of the outer tube is further needed, so that the guide wire can extend out through the opening of the outer tube. However, when releasing the stent, the inner tube needs to slide relative to the outer tube, and the guide wire outlet of the inner tube intersects with the opening of the side wall of the outer tube. The guide wire will be clamped and fixed between the inner tube and the outer tube, which will lead to the technical problem that the stent cannot be released smoothly.

### SUMMARY

The object of the present disclosure is to provide a rapid exchange stent delivery device to solve the technical problems in the prior art in which the operation of the stent delivery device is time-consuming and the release of the stent is prone to jamming.

In a first aspect, the present disclosure provides a rapid exchange stent delivery device for releasing a stent toward a distal end along a guide wire, and the delivery device includes: an outer tube and an inner tube inserted inside the outer tube;
a guide wire inlet is arranged on a distal end of the inner tube, and a guide wire outlet is arranged on a side wall; and
the outer tube has an avoiding part extending toward the distal end to avoid and guide out the guide wire extending from the guide wire outlet, so that the outer tube can move relative to the inner tube, and the inner tube pushes the stent out of the distal end of the outer tube for release.

In conjunction with the first aspect, the present disclosure provides one possible embodiment of the first aspect, wherein the avoiding part includes an avoiding sliding slot, and the avoiding sliding slot is arranged on a side wall of the outer tube and extends along an axial direction of the outer tube.

In conjunction with one possible embodiment of the first aspect, the present disclosure provides another possible embodiment of the first aspect, wherein the outer tube includes: a tube body segment and a flat body segment connected to a proximal end of the tube body segment;
the flat body segment has an arc shape in a section perpendicular to an axis of the outer tube, and the flat body segment is located on a side of the inner tube away from the guide wire outlet.

In conjunction with the first aspect, the present disclosure provides another possible embodiment of the first aspect, wherein the outer tube includes: a first tube segment and an opening segment connected to a proximal end of the first tube segment, wherein the first tube segment is sleeved on the inner tube; and
the avoiding part includes: a gap part located between the first tube segment and the inner tube, and an opening part arranged on the opening segment.

In conjunction with another possible embodiment of the first aspect, the present disclosure provides another possible embodiment of the first aspect, wherein the opening part has a first guiding surface slanting from the distal end toward the proximal end and toward the outer side of the guide wire outlet.

In conjunction with another possible embodiment of the first aspect, the present disclosure provides another possible embodiment of the first aspect, wherein the proximal end of the opening segment is connected to a second tube segment sleeved on the inner tube.

In conjunction with another possible embodiment of the first aspect, the present disclosure provides another possible embodiment of the first aspect, wherein an inner diameter of the first tube segment is larger than or equal to an inner diameter of the second tube segment.

In conjunction with the first aspect, the present disclosure provides another possible embodiment of the first aspect, wherein a guiding part is arranged inside the inner tube, and the guiding part is located on the proximal end of the guide wire outlet.

In conjunction with the first aspect, the present disclosure provides another possible embodiment of the first aspect, wherein the guiding part has a second guiding surface, and the second guiding surface is an inclined surface slanting from the distal end toward the proximal end and toward an outer side of the guide wire outlet.

In conjunction with the first aspect, the present disclosure provides another possible embodiment of the first aspect, wherein the inner tube is connected to a pushing member; the pushing member is inserted inside the outer tube; and the pushing member is configured to push the stent out from the distal end of the outer tube for release.

In conjunction with the first aspect, the present disclosure provides another possible embodiment of the first aspect, wherein the rapid exchange stent delivery device further includes a retrieval member; the retrieval member is arranged on a distal side of the pushing member; and the retrieval member is configured to retrieve the released the stent back into the outer tube.

In conjunction with the first aspect, the present disclosure provides another possible embodiment of the first aspect, wherein a side wall of the outer tube is provided with an avoiding slot extending from the avoiding part to the proximal end; and
when the inner tube slides toward the proximal end relative to the outer tube, the guide wire extending from the guide wire outlet slides toward the proximal end along the avoiding slot.

In conjunction with the first aspect, the present disclosure provides another possible embodiment of the first aspect, wherein the proximal ends of the outer tube and the inner tube are respectively connected to an operation handle, and the operation handle is configured to drive the inner tube to slide relative to the outer tube.

In conjunction with the first aspect, the present disclosure provides another possible embodiment of the first aspect, wherein the rapid exchange stent delivery device further includes a supporting member; the supporting member is arranged in the inner tube; and a distal end of the supporting member extends no further than the guide wire outlet.

In conjunction with the first aspect, the present disclosure provides another possible embodiment of the first aspect, wherein the stent is fixedly bounded to a distal end of the pushing member or an outer surface of the inner tube by at least one fixing wire.

In conjunction with the first aspect, the present disclosure provides another possible embodiment of the first aspect, wherein a diameter of the inner tube gradually increases from the distal end to the proximal end; or
a wall thickness of the outer tube gradually increases from the distal end to the proximal end.

The embodiments of the present disclosure include the following beneficial effects. It adopts the inner tube with a guide wire inlet at the distal end; a guide wire outlet is arranged at the side wall of the inner tube; and the inner tube is inserted into the outer tube. The outer tube has an avoiding part extending toward the distal end to avoid and guide out the guide wire extending from the guide wire outlet, so that the outer tube can move relative to the inner tube, and the inner tube pushes the stent out of the distal end of the outer tube for release, which shortens the length of the guide wire extending to the proximal end of the inner tube to realize the rapid exchange, and shortens the operation duration of the surgery. This avoids the guide wire from being jammed between the outer tube and the inner tube, thereby ensuring that the stent can be smoothly released.

In order to make the above objectives, features, and advantages of the present disclosure more obvious and easier to understand, the following is a detailed description of preferred embodiments in conjunction with the drawings.

### BRIEF DESCRIPTION OF DRAWINGS

In order to more clearly illustrate the technical solutions in the specific embodiments or related technologies of the present disclosure, the drawings to be used in the description of the specific embodiments or related technologies will be briefly introduced below, and it will be obvious that the drawings in the following description are some of the embodiments of the present disclosure, and that for a person of ordinary skill in the field, other drawings can also be obtained based on these drawings without any inventive effort.

FIG. 1 shows a local schematic diagram of an outer tube, an inner tube, and a guide wire of a rapid exchange stent delivery device provided by the embodiments of the present disclosure;

FIG. 2 shows a sectional diagram of a distal end of a rapid exchange stent delivery device provided by the embodiments of the present disclosure;

FIG. 3 shows a sectional diagram of a rapid exchange stent delivery device at a guide wire outlet provided by the embodiments of the present disclosure;

FIG. 4 shows a schematic diagram of an outer tube of a rapid exchange stent delivery device provided by the embodiments of the present disclosure;

FIG. 5 shows a schematic diagram of an outer tube, an inner tube, and a stent of another rapid exchange stent delivery device provided by the embodiments of the present disclosure;

FIG. 6 shows a sectional diagram of another rapid exchange stent delivery device provided by the embodiments of the present disclosure; and

FIG. 7 shows a schematic diagram of a rapid exchange stent delivery device provided by the embodiments of the present disclosure.

Reference numbers: 100—outer tube; 011—tube body segment; 012—flat body segment; 013—first tube segment; 014—opening segment; 015—second tube segment; 110—avoiding part; 111—avoiding sliding slot; 112—gap part; 113—opening part; 200—inner tube; 201—guide wire inlet; 202—guide wire outlet; 203—guiding part; 300—guide wire; 400—stent; 500—pushing member; 600—operation handle; 700—supporting member.

### DETAILED DESCRIPTION OF EMBODIMENTS

The technical solutions in the embodiments of the present disclosure will be clearly and completely described below in conjunction with the drawings in the embodiments of the present disclosure. It is obvious that the embodiments described are partial embodiments of the present disclosure, and not all of the embodiments. Based on the embodiments in the present disclosure, all other embodiments obtained by a person of ordinary skill in the art without inventive efforts shall fall within the scope of protection of the present disclosure.

In the description of the present disclosure, it is to be noted that orientation or positional relationships indicated by terms, such as “center”, “up”, “down”, “left”, “right”, “vertical”, “horizontal”, “inside”, “outside”, are the orientation or positional relationships based on the drawings, which are only to facilitate the description of the present disclosure and simplify the description, and are not to indicate or imply that the device or element referred to must have a particular orientation, or be constructed and operated with a particular orientation, and are not to be understood as limitations of the present disclosure. The “distal end” and “proximal end” are defined based on the operating end or the person, wherein the end that is farther is “distal end”, and the end that is closer is “proximal end”. Additionally, the terms “first”, “second”, and “third”, etc., are only used to describe the differences in names, and are not to be understood as indicating or implying a relative importance. The physical quantities in the formula, if not separately marked, should be understood as the base quantities of the base units in the International System of Units, or the derived quantities derived from the base quantities through mathematical operations such as multiplication, division, differentiation or integration.

In the description of the present disclosure, it should be noted that unless other expressly specifications and limitations, the terms “mount”, “connect”, and “link” are to be understood in a broad sense. For example, it can be a fixed connection, a detachable connection, or an integral connection; it can be a mechanical connection or an electrical connection; and it can be a direct connection, an indirect connection through an intermediate medium, or a communication inside two components. For a person of ordinary skill in the art, the specific meaning of the above terms in the present disclosure can be understood in specific situations.

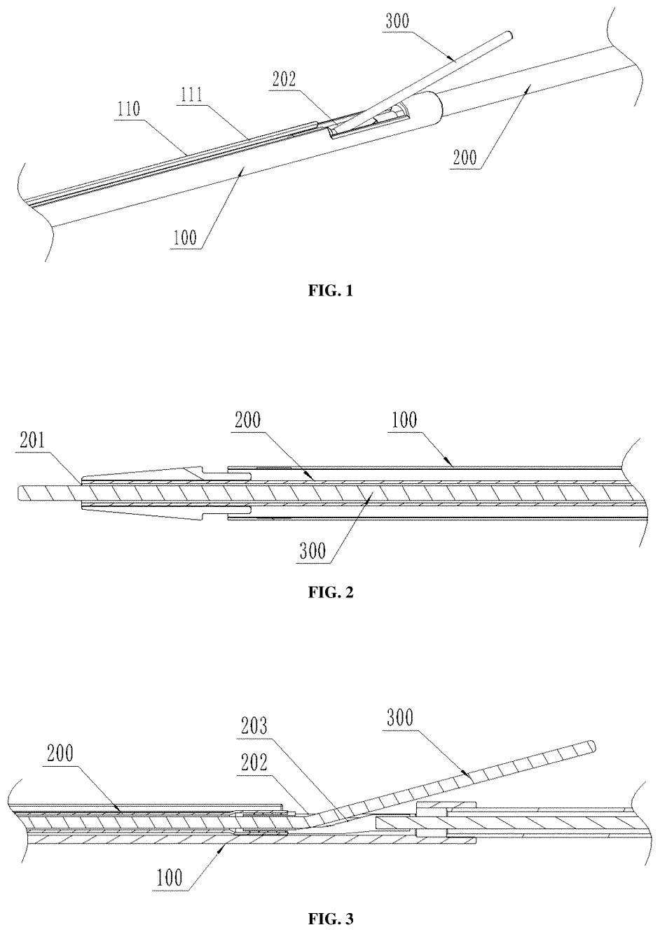

As shown in FIG. 1, FIG. 2, and FIG. 5, the embodiment of the present disclosure provides a rapid exchange stent delivery device for releasing a stent 400 toward a distal end along a guide wire 300, and the delivery device includes: an outer tube 100 and an inner tube 200 inserted inside the outer tube 100, wherein a guide wire inlet 201 is arranged on a distal end of the inner tube 200, and a guide wire outlet 202 is arranged on a side wall of the inner tube 200. The outer tube 100 has an avoiding part 110 extending toward the distal end and configured to avoid and guide out the guide wire 300 extending from the guide wire outlet 202, so that the outer tube 100 can move relative to the inner tube 200, and the inner tube 200 pushes the stent 400 out of the distal end of the outer tube 100 for release.

When the stent 400 is conveyed, the outer tube 100 and the inner tube 200 simultaneously move to the distal end along the guide wire 300, so that the stent 400 inside the outer tube 100 is conveyed to a preset position. When the stent 400 needs to be released, the inner tube 200 pushes the stent 400 to the distal end relative to the outer tube 100, so that the stent 400 is pushed out from the distal end of the outer tube 100, and the released stent 400 can expand radially through its own elasticity. When the inner tube 200 moves to the distal end relative to the outer tube 100, the guide wire 300 extending from the guide wire outlet 202 slides to the distal end along the avoiding part 110, thereby avoiding the guide wire 300 from being jammed between the outer tube 100 and the inner tube 200, so as to ensure a smooth release of the stent 400. In addition, the guide wire 300 extending from the guide wire outlet 202 can be led out via the avoiding part 110, which shortens the length of the guide wire 300 extending to the proximal end of the outer tube 100 and thus shortens the operating duration for exchanging the stent.

In another embodiment, when the stent 400 is conveyed, the outer tube 100 and the inner tube 200 simultaneously move to the distal end along the guide wire 300, so that the stent 400 inside the outer tube 100 is conveyed to a preset position. When it needs to release the stent 400, the outer tube 100 moves to the proximal end relative to the inner tube 200, the inner tube 200 pushes against the stent 400 to release it from the distal end of the outer tube 100, and the released stent 400 can expand radially through its own elasticity. When the outer tube 100 moves towards the proximal end relative to the inner tube 200, the avoiding part 110 slides toward the proximal end along the guide wire 300 extending from the guide wire outlet 202, thereby preventing the guide wire 300 from being jammed between the outer tube 100 and the inner tube 200.

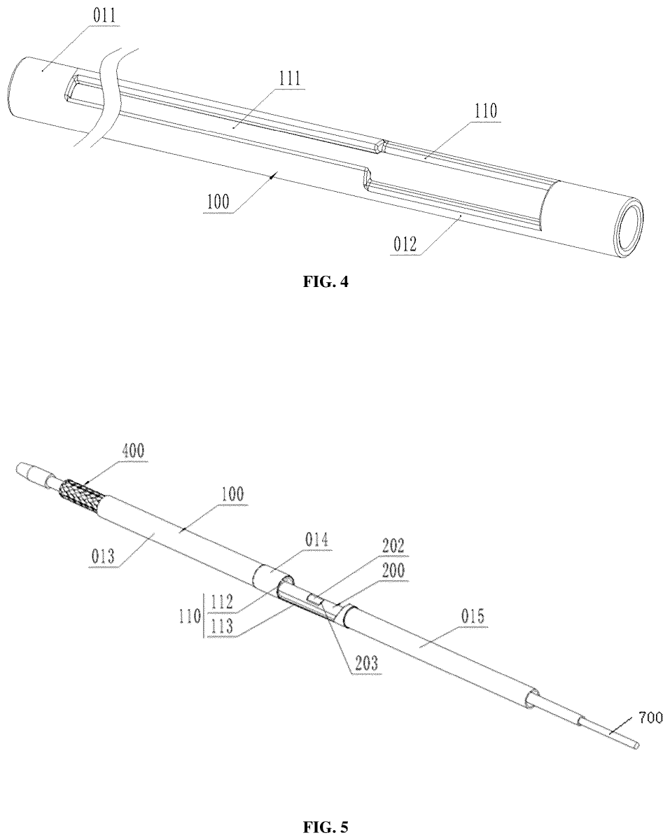

As shown in FIG. 1 and FIG. 4, in the embodiment of the present disclosure, the avoiding part 110 includes an avoiding sliding slot 111, and the avoiding sliding slot 111 is arranged on a side wall of the outer tube 100 and extends along an axial direction of the outer tube 100.

The proximal end of the avoiding sliding slot 111 corresponds to the guide wire outlet 202, and the avoiding sliding slot 111 extends toward the distal end along the side wall of the outer tube 100. When the inner tube 200 moves toward the distal end relative to the outer tube 100 or the outer tube 100 moves toward the proximal end relative to the inner tube 200, the guide wire 300 extending from the guide wire outlet 202 can slide relative to the avoiding sliding slot 111, so as to ensure that the inner tube 200 can slide smoothly relative to the outer tube 100.

Referring to FIG. 1 and FIG. 4, the outer tube 100 includes: a tube body segment 011 and a flat body segment 012 connected to a proximal end of the tube body segment 011, wherein the flat body segment 012 has an arc shape in a section perpendicular to an axis of the outer tube 100, and the flat body segment 012 is located on a side of the inner tube 200 away from the guide wire outlet 202. Therefore, a larger opening is formed on a side of the flat body segment 012 facing the guide wire 300 extending from the guide wire outlet 202. The guide wire 300 can be introduced outward through the opening, and can also slide toward the distal end along the opening, so that the inner tube 200 can slide smoothly relative to the outer tube 100.

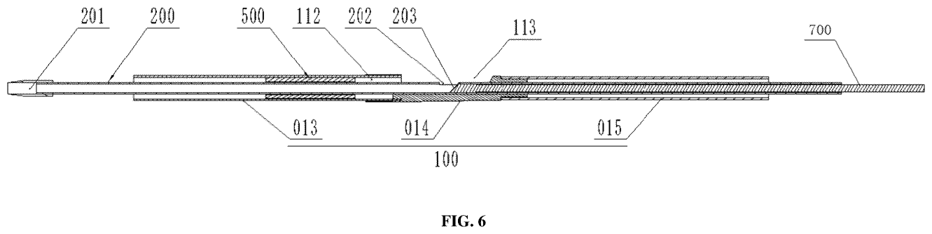

In another embodiment, referring to FIG. 2, FIG. 5, and FIG. 6, the outer tube 100 includes: a first tube segment 013 and an opening segment 014 connected to a proximal end of the first tube segment 013. The first tube segment 013 is sleeved on the inner tube 200. The avoiding part 110 includes: a gap part 112 located between the first tube segment 013 and the inner tube 200, and an opening part 113 arranged on the opening segment 014.

Specifically, the inner diameter of the first tube segment 013 is larger than the outer diameter of the inner tube 200, so that a gap part 112 can be formed between the first tube segment 013 and the inner tube 200. The guide wire 300 extending from the guide wire outlet 202 can be led out via the opening part 113. When the inner tube 200 moves toward the distal end relative to the outer tube 100 or the outer tube 100 moves toward the proximal end relative to the inner tube 200, the guide wire 300 extending from the guide wire outlet 202 can slide into the gap part 112 via the opening part 113. Therefore, the inner tube 200 and the outer tube 100 will not be jammed during sliding, so as to ensure the smooth releasing of the stent 400.

Optionally, the opening part 113 has a first guiding surface slanting from the distal end toward the proximal end and toward the outer side of the guide wire outlet 202. The first guiding surface is configured to change a direction of the guide wire 300 extending from the guide wire outlet 202 to prevent the guide wire 300 from being jammed between the guide wire outlet 202 and the outer tube 100.

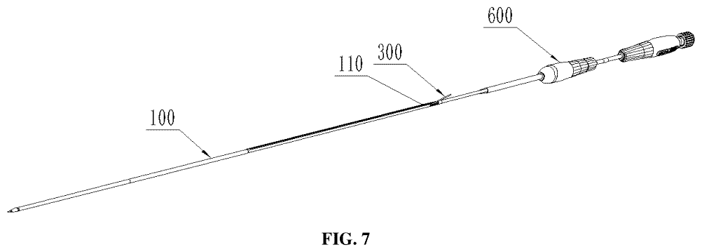

Further, a second tube segment 015 sleeved on the inner tube 200 is connected to the proximal end of the opening segment 014. Referring to FIG. 5 and FIG. 7, the second tube segment 015 extends to the proximal end and is connected to the operation handle 600.

In an optional embodiment, an avoiding slot capable of accommodating sliding of the guide wire 300 can be arranged on the side wall of the second tube segment 015. When the inner tube 200 slides to the proximal end relative to the outer tube 100, the guide wire 300 extending from the guide wire outlet 202 slides to the proximal end along the avoiding slot.

As shown in FIG. 5 and FIG. 6, the inner diameter of the first tube segment 013 is larger than or equal to the inner diameter of the second tube segment 015, and the second tube segment 015 is in clearance fit with the inner tube 200, thereby ensuring that the inner tube 200 can slide smoothly along the outer tube 100.

The stent 400 in the embodiment is arranged between the inner tube 200 and the outer tube 100. Optionally, the stent 400 can be fixedly bounded to the outer surface of the inner tube 200 by at least one fixing wire. When the outer tube 100 removes the first tube segment 013, and the stent 400 is exposed or partially exposed to the external environment, the stent 400 can be connected to the pushing member 500 or the inner tube 200 by the fixing wire, so that the stent 400 keeps in a contracted state when not released. When the stent 400 is allowed to release, the stent 400 is released and unfolded by pulling the fixing wire. Optionally, the distal end of the pushing member 500 is connected to the stent 400 by at least one fixing wire. Of course, the pushing member 500 can only abut against the stent 400 without being connected to the stent 400, which is not limited herein.

Optionally, in order to ensure the pushing force of the inner tube 200 and to prevent jamming during relative sliding between the inner tube 200 and the outer tube 100, in the embodiment, a diameter of the inner tube 200 gradually increases from the distal end to the proximal end; or a wall thickness of the outer tube 100 gradually increases from the distal end to the proximal end. In the embodiment, the diameter of the distal end of the inner tube 200 is smaller than the diameter of the proximal end of the inner tube 200, i.e., the end of the inner tube 200 close to the operation handle 600 is thicker and the end of the inner tube 200 away from the operation handle 600 is thinner. Of course, the localized thickening or step thickening can be performed on the distal end of the inner tube 200, which is not limited herein. The stepped thickening refers to the gradual thickening of the inner tube 200 from the distal end to the proximal end. The diameter of the inner tube 200 can also be uniform throughout, which is not limited herein. When the diameter of the outer tube 100 is unchanged, the wall thickness of the outer tube gradually increases from the distal end to the proximal end, and the outer tube 100 can also be locally thickened only at the proximal end, which is not limited herein.

As shown in FIG. 3, FIG. 5, and FIG. 6, in an optional embodiment, the inner tube 200 is provided with a guiding part 203 inside, wherein the guiding part 203 is located at the proximal end of the guide wire outlet 202 and has an inclined surface slanting from the distal end toward the proximal end and toward an outer side of the guide wire outlet 202.

The guide wire 300 enters from the guide wire inlet 201 at the distal end of the inner tube 200 and extends toward the guide wire outlet 202. The guide wire 300 extending to the guide wire outlet 202 can be inclined toward the outer side along the inclined surface of the guiding part 203, so that the guide wire 300 can be smoothly guided out.

The guiding part 203 has a second guiding surface, and the second guiding surface is an inclined surface slanting from the distal end toward the proximal end and toward an outer side of the guide wire outlet 202. The second guiding surface is configured to lead the guide wire 300 out of the guide wire outlet 202 to prevent the guide wire 300 from being jammed between the inner tube 200 and the outer tube 100. The second guiding surface can be machined and molded directly by a material of the tube wall of the inner tube 200, e.g., it can be formed into an inclined surface through processes such as hot molding, laser cutting, or mechanical cutting, so that the guiding wire 300 can naturally slide out of the guide wire outlet 202 along the guiding surface.

Optionally, the second guiding surface shell is formed by filling with glue and curing. After the glue is cured, it forms a smooth transition inclined surface, which can reduce the resistance when the guide wire 300 passes through. Optionally, the second guiding surface can also be composed of an independently manufactured inclined surface component, which is fixedly embedded in the inner tube 200 and is close to the guide wire outlet 202. The inclined surface component can be fixed to the inner tube 200 by bonding or interference fit, etc. The second guiding surface can be made of metal or polymer material, etc., which is not limited herein.

Referring to FIG. 5 and FIG. 6, the inner tube 200 is connected to a pushing member 500; the pushing member 500 is inserted inside the outer tube 100; and the pushing member 500 is configured to push the stent 400 out from the distal end of the outer tube 100 for release. When the inner tube 200 slides toward the distal end along the outer tube 100 or the outer tube 100 moves toward the proximal end relative to the inner tube 200, the inner tube 200 drives the pushing member 500 to slide toward the distal end, and the pushing member 500 pushes the stent 400 out from the distal end of the outer tube 100 for release.

Optionally, the rapid exchange stent delivery device in the embodiments further includes a retrieval member; the retrieval member is arranged on a distal side of the pushing member 500; and the retrieval member is configured to retrieve the released the stent 400 back into the outer tube 100. Optionally, the retrieval member is configured to drive the released stent to move in a direction opposite to the pushing direction, thereby retracting the released stent 400 back into the outer tube 100. Optionally, the pushing member is connected to the retrieval member; one end of the retrieval member away from the pushing member 500 is connected to the stent 400; and the retrieval member is configured to retrieve the released stent 400 back into the outer tube 100. Of course, the stent 400 can also be arranged to cover the retrieval member, which is not limited herein. When the stent 400 is released and needs to be adjusted or retrieved, a retraction force is applied by the pushing member 500, so that the retrieval member drives the stent 400 to move toward the proximal end and to re-enter the tube cavity of the outer tube 100. Optionally, the retrieval member can be a flexible pulling wire, metal wire, etc., and the distal end is connected to the stent 400. The shape and material of the retrieval member are not limited herein, and only need to facilitate retrieval of the stent 400. The retrieval member can be connected to the stent 400 by using connection methods such as a sleeve ring, snap-fit, bonding, or wire winding, and the connection method between the retrieval member and the stent 400 and the pushing member 500 is not limited herein.

Optionally, in order to facilitate the retrieval of the stent 400, the distal end of the outer tube 100 can be provided with a conical or flared structure.

As shown in FIG. 3 and FIG. 6, in an optional embodiment, a side wall of the outer tube 100 is provided with an avoiding slot extending from the avoiding part 110 to the proximal end. When the inner tube 200 slides to the proximal end relative to the outer tube 100, the guide wire 300 extending from the guide wire outlet 202 slides to the proximal end along the avoiding slot.

As shown in FIG. 2, FIG. 5, and FIG. 7, the proximal ends of the outer tube 100 and the inner tube 200 are respectively connected to an operation handle 600, and the operation handle is configured to drive the inner tube 200 to slide relative to the outer tube 100. Additionally, in an optional embodiment, the operation handle 600 can further be provided with a snap or threaded locking structure for locking the inner tube 200 relative to the outer tube 100 and unlocking them only when releasing the stent 400. The operation handle 600 is used to drive the inner tube 200 to slide toward the distal end relative to the outer tube 100, so that the stent 400 is released from the distal end of the outer tube 100.

Optionally, the rapid exchange stent delivery device in the embodiment further includes a supporting member; the supporting member 700 is arranged in the inner tube 200; and a distal end of the supporting member 700 extends no further than the guide wire outlet 202. Optionally, one end of the supporting member in the embodiment is connected to the operation handle 600, and the other end of the supporting member extends towards the distal end and extends into the inner tube 200. The supporting member provides radial rigidity for the inner tube 200 to prevent the inner tube 200 from collapsing or undergoing excessive deformation during the pushing process, which ensures that the guide wire 300 can move normally within the inner tube 200, and ensures the smooth release or retrieval of the stent 400. The bending resistance of the inner tube 200 can also be enhanced by providing the supporting member, which ensures that the pushing force can be effectively transmitted to the distal end, thereby improving the accuracy of the operation. In order to avoid affecting the entry and exit of the guide wire 300 through the guide wire outlet 202, and to prevent the guiding wire 300 from being jammed between the inner tube 200 and the outer tube 100, the end of the supporting member away from the operation handle 600 extends no further than the guide wire outlet 202.

The supporting member in the embodiment is a supporting wire. Of course, the supporting member can also be designed in other shapes and structures, which are not limited herein. In order to meet the support requirements of different segments of the inner tube 200, the rigidity of the supporting member can be uniformly distributed or gradually changed in distribution along the length direction. For example, the diameter of the supporting member gradually decreases from the end close to the operation handle 600 to the end far from the operating handle 600; or the middle part of the supporting member is thickened, etc., which is not limited herein.

Optionally, the supporting member in the embodiment is threadedly connected to the operation handle 600. Of course, the supporting member can be connected to the operation handle 600 by methods such as snap connection, compressing connection, and wire winding, which is not limited herein.

Finally, it should be noted that the above embodiments are only used to illustrate the technical solutions of the present disclosure, and not to restrict it. Although the present disclosure is described in detail with reference to the foregoing embodiments, it should be understood by persons of ordinary skill in the art that they can still modify the technical solutions described in the foregoing embodiments or make equivalent substitutions for some or all of the technical features, and these modifications or substitutions do not take the essence of the corresponding technical solutions out of the scope of the technical solutions in each embodiment of the present disclosure.

1. A rapid exchange stent delivery device, configured to release a stent to a distal end along a guide wire, wherein the rapid exchange stent delivery device comprises:
an outer tube;
an inner tube inserted inside the outer tube;
a guide wire inlet arranged on a distal end of the inner tube; and
a guide wire outlet is arranged on a side wall,
wherein the outer tube has an avoiding part extending toward a distal end to avoid and guide out the guide wire extending from the guide wire outlet, so that the outer tube is allowed to move relative to the inner tube, and the inner tube pushes the stent out of the distal end of the outer tube for release.

2. The rapid exchange stent delivery device of claim 1, wherein the avoiding part comprises an avoiding sliding slot, and the avoiding sliding slot is arranged on a side wall of the outer tube and extends along an axial direction of the outer tube.

3. The rapid exchange stent delivery device of claim 2, wherein the outer tube comprises: a tube body segment and a flat body segment connected to a proximal end of the tube body segment; and
the flat body segment has an arc shape in a section perpendicular to an axis of the outer tube, and the flat body segment is located on a side of the inner tube away from the guide wire outlet.

4. The rapid exchange stent delivery device of claim 1, wherein the outer tube comprises: a first tube segment, and an opening segment connected to a proximal end of the first tube segment; the first tube segment is sleeved on the inner tube; and
the avoiding part comprises: a gap part located between the first tube segment and the inner tube, and an opening part arranged on the opening segment.

5. The rapid exchange stent delivery device of claim 4, wherein the opening part has a first guiding surface slanting from a distal end of the guide wire outlet toward a proximal end of the guide wire outlet and toward an outer side of the guide wire outlet.

6. The rapid exchange stent delivery device of claim 4, wherein a proximal end of the opening segment is connected to a second tube segment sleeved on the inner tube (200).

7. The rapid exchange stent delivery device of claim 6, wherein an inner diameter of the first tube segment is larger than or equal to an inner diameter of the second tube segment.

8. The rapid exchange stent delivery device of claim 1, wherein a guiding part is arranged inside the inner tube, and the guiding part is located on a proximal end of the guide wire outlet.

9. The rapid exchange stent delivery device of claim 8, wherein the guiding part has a second guiding surface, and the second guiding surface is an inclined surface slanting from a distal end of the guide wire outlet toward the proximal end of the guide wire outlet and toward an outer side of the guide wire outlet.

10. The rapid exchange stent delivery device of claim 1, wherein the inner tube is connected to a pushing member; the pushing member is inserted inside the outer tube; and the pushing member is configured to push the stent out from the distal end of the outer tube for release.

11. The rapid exchange stent delivery device of claim 10, wherein the rapid exchange stent delivery device further comprises a retrieval member; the retrieval member is arranged on a distal side of the pushing member; and the retrieval member is configured to retrieve the released stent back into the outer tube.

12. The rapid exchange stent delivery device of claim 1, wherein a side wall of the outer tube is provided with an avoiding slot extending from the avoiding part to a proximal end; and
when the inner tube slides toward the proximal end relative to the outer tube, the guide wire extending from the guide wire outlet slides toward the proximal end along the avoiding slot.

13. The rapid exchange stent delivery device of claim 1, wherein proximal ends of the outer tube and the inner tube are respectively connected to an operation handle, and the operation handle is configured to drive the inner tube to slide relative to the outer tube.

14. The rapid exchange stent delivery device of claim 1, wherein the rapid exchange stent delivery device further comprises a supporting member; the supporting member is arranged in the inner tube; and a distal end of the supporting member extends no further than the guide wire outlet.

15. The rapid exchange stent delivery device of claim 1, wherein the stent is fixedly bounded to a distal end of a pushing member or an outer surface of the inner tube by at least one fixing wire.

16. The rapid exchange stent delivery device of claim 1, wherein a diameter of the inner tube gradually increases from the distal end to a proximal end.

17. The rapid exchange stent delivery device of claim 1, wherein a wall thickness of the outer tube gradually increases from the distal end to a proximal end.
